(19) United States
(12) Patent Application Publication
(10) Pub. No.: US 2008/0125792 A1
(43) Pub. Date: May 29, 2008

(54) Vacuum Extractor And Method Of Use Therefor

(75) Inventors: Daniel Anthony Giardina, New Fairfield, CT (US); Allen Eugene Brandenburg, Dripping Springs, TX (US)

Correspondence Address: LATHROP & GAGE LC, 2345 GRAND AVENUE, SUITE 2800, KANSAS CITY, MO, 64108, US

(73) Assignee: COOPERSURGICAL, INC., Trumbull, CT (US)

(21) Appl. No.: 11/942,156

(22) Filed: Nov. 19, 2007

Related U.S. Application Data
(60) Provisional application No. 60/861,599, filed on Nov. 29, 2006.

(51) Int. Cl. A61B 17/42

(52) U.S. Cl. 606/123

(57) ABSTRACT

A vacuum extractor apparatus for assisting in childbirth includes a cup and a stem for connection to a vacuum source, that pulls a vacuum through the apparatus. There is a joint between the cup and the stem, the joint being such that bending of the stem, or bending of the cup in relation to the stem, does not occlude the vacuum channel or pathway, and allows the vacuum to be maintained, or controlled.

### CROSS-REFERENCES TO RELATED APPLICATIONS

This application is related to and claims priority from U.S. Provisional Patent Application Ser. No. 60/861,599, entitled: Vacuum Extractor and Method of Use Therefor, filed Nov. 29, 2006, the disclosure of which is incorporated by reference herein.

### TECHNICAL FIELD

The disclosed subject matter is directed to apparatus for facilitating the extraction of children from the birth canal, and in particular, to vacuum extractors.

### BACKGROUND

Childbirth is typically a natural process. However, there are many instances that the process of childbirth must be assisted by instrumentation. For example, the mother may be too weak or incapacitated to continue with the labor effort. Alternately, the child may be too weak to sustain a long labor. In both cases, rapid delivery is necessary.

Additionally, the labor may simply be too weak or there may be insufficient effort by the mother. The child's positioning in the birth canal may also result in assisted delivery.

Vacuum extractors are one type of instrumentation, that is used in assisted delivery. Typical vacuum extractors are formed of a cup, with a bell or mushroom shape, with an wide open end for receiving and engaging the head of the child, and a smaller open end for receiving a stem, to which a vacuum source is attached.

The choice of assisted delivery with a bell or mushroom shaped cup on a vacuum extractor is made by the physician at the time of delivery. As a general rule, bell shaped cup extractors appear to be more appropriate for use for uncomplicated occipital anterior extractions, while mushroom shaped cup extractors are more suitable for occipital posterior, occipital transverse, and difficult occipital anterior position deliveries.

An exemplary vacuum extractor with a mushroom shaped cup is disclosed in U.S. Pat. No. 5,019,086 (Neward). This vacuum extractor includes a stem, with a section proximate to the connection to the cup, that is relatively thin walled. This thin walled section provides a flexible portion to the stem, and allows the cup to be bent or folded to a position parallel to the stem. Specifically, the bend occurs at a pinch point along the flexible portion of the stem. The flexibility of the flexible portion is such that once the cup is brought to its desired position, it is released, such that the stem springs back to its original position, in axial alignment with the cup.

This construction exhibits numerous drawbacks. First, the pinch point cuts off or severely limits the vacuum, and once there is a pinch, it is difficult to apply more vacuum. With the vacuum “pinched off”, the cup may “pop off”, the head of the child, an unintended result. Additionally, such pinching makes maneuvering the cup with the stem difficult. Moreover, once a pinch in the stem occurs, the stem expands laterally creating sharp edges, that may cause trauma to the tissues of the birth canal.

### SUMMARY

The disclosed extractor and method for its use improve on the contemporary art. The disclosed extractor is such that it can follow the vaginal walls and remain parallel thereto, to compensate for asynclitism. The disclosed extractor is such that it can maintain the position of the cup as its movement is guided by changing angles of the vaginal walls of the birth canal. The disclosed extractor also includes a joint between the cup and the stem, the joint being such that bending of the stem, or bending of the cup in relation to the stem, does not occlude the vacuum channel or pathway, and allows the vacuum to be maintained, or controlled, as it is pulled through the apparatus.

The joint is also flexible, such that upon the aforementioned bending, it remains rounded, and “tissue friendly”, as sharp edges do not form. Additionally, the flexibility of the joint allows the cup and stem to return to their initial positions gradually, as opposed to snapping back to the initial position, once the pressure causing the bend is released. As a result of this structure, the doctor or clinician maintains maximum control over the head of the child during delivery, with the apparatus at various desired angles.

The disclosed subject matter is directed to a vacuum extractor apparatus. The apparatus has a cup with a first open end, for gripping and sealing with the head of a child in the birth canal, and a second open end, and a stem. The stem has a first end, for coupling with the second end of the cup, and, a second end for coupling with a vacuum source. The cup is coupled to the stem by a flexible joint. This joint is constructed to bend, in order to allow movement of at least one of the cup or the stem. The bending is, for example, such that the axes of the cup and stem may be oriented with respect to each other, for movement between positions where the cup and stem axes are perpendicular, or substantially perpendicular to each other, to positions where the cup and stem axes are parallel to each other, or substantially parallel to each other, and all angled orientations of the axes and corresponding positions of the cup and stem therebetweeen. At all of these positions, and orientations of the cup axis and stem axis with respect to each other, during the bending of the joint, the inside of the joint is open to maintain a pathway for a vacuum through the apparatus, from the cup to the stem.

The flexible joint is such that its outer surface forms a smooth transition with the outer surfaces of cup. The cup is for example, of a mushroom-like shape. The flexible joint is, for example, of a material of greater flexibility than the material of the cup.

Another embodiment is directed to a vacuum extractor apparatus. The apparatus includes a cup, a stem, and a joint for securing the cup to the stem. The cup includes a first open end and a second open end. The stem includes a first end for coupling with the second end of the cup, and, a second end for coupling with a vacuum source. The joint is configured for accommodating bending of the apparatus at the joint at least at a pinch point, and it is also configured to accommodate the bending while maintaining a vacuum, that is, for example, being pulled, through the apparatus. The cup is, for example, mushroom shaped.

The apparatus may also accommodate either a handle at the stem, and connect to a vacuum source, or couple to a hand pump (at the stem) that provides (pulls) a vacuum in the apparatus. The hand pump couples with the stem so as to be rotatable independent of the stem.

Another embodiment is directed to a vacuum extractor apparatus, including a cup, a stem, and a joint for coupling the cup to the stem. The cup includes a cup with a first open end and a second open end. The stem includes a first end in communication with the second end of the cup, and, a second end for communication with a vacuum source, and the joint accommodates bending of the apparatus at the joint, at least at a pinch point. The joint is designed to bend such that its outer surface remains smooth, free of sharp edges, greatly reducing chances for trauma to maternal tissues.

Another embodiment is directed to a method for making a vacuum extractor apparatus. The method includes, providing a cup, for example, a mushroom-shaped cup, and a stem operatively coupled with the cup, so as to be at least substantially coaxial. A joint of a flexible material, for example, of greater flexibility than the material of at least the cup, that is, for example, of a semi-rigid material, is molded onto the cup and the stem. The cup and the stem may be a unitary member or separate pieces.

Another embodiment of the disclosed subject matter is directed to a method for assisting in the extraction of a child during childbirth. The method includes initially providing an extraction apparatus, that includes, a cup with a first open end and a second open end, and a stem. The stem includes a first end that is operatively coupled to the second end of the cup, and, a second end for being operatively coupled to a vacuum source. A joint attaches to the cup and stem. The joint accommodates bending of the apparatus, at the joint, at least at a pinch point. The joint also accommodates bending while maintaining a vacuum through (being pulled through) the apparatus. In no particular order, the stem is connected to a vacuum source, and moved to cause the joint to bend. The cup is inserted into the birth canal into contact with the head of a child. If desired, the vacuum source may also be connected at this point. The vacuum source is activated and the stem is moved to maneuver the child through the birth canal. The vacuum source may be, for example, a conventional suction source, used to pull a vacuum, or a hand pump, attached to the stem.

### BRIEF DESCRIPTION OF THE DRAWINGS

Attention is now directed to the drawing figures, where like or corresponding numerals indicate like or corresponding components. In the drawings:

FIG. 1 is a side view of the apparatus of the disclosed subject matter in a first position;

FIG. 2 is a side view of the distal or “cup” portion end of the apparatus of FIG. 1 in a first position;

FIG. 3 is a side view of the distal or “cup” portion end of the apparatus of FIG. 1 in a second position;

FIG. 4 is a bottom view of the apparatus of FIG. 1;

FIG. 5 is a cross-sectional view of the apparatus of FIG. 4, taken along line 5-5;

FIG. 6 is the cross sectional view of FIG. 5 showing the vacuum in the apparatus, when the apparatus is in the first position;

FIG. 7 is the cross sectional view of FIG. 3 along line 7-7, showing the vacuum in the apparatus; and,

FIG. 8 is a side view of an alternate apparatus of the disclosed subject matter in a first position.

### DETAILED DESCRIPTION OF THE DRAWINGS

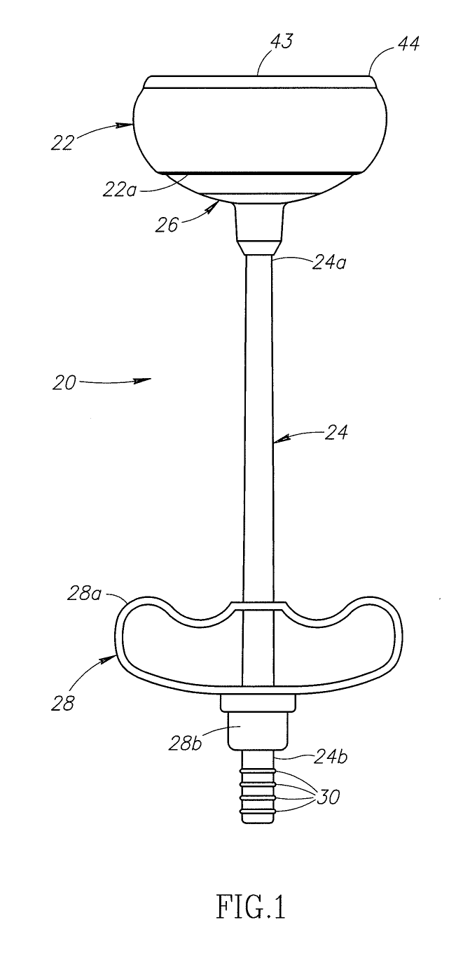

FIG. 1 shows an apparatus 20, a vacuum extractor. The apparatus 20 includes a vacuum cup 22 and a stem 24, with a joint 26, for coupling the cup 22 to the stem 24. The stem 24 is hollow, and includes a distal end 24a, where the joint 26 attaches to the base 22a of the cup 22, and a proximal end 24b, where a handle 28, formed of a handle portion 28a and a tail cap 28b, is attached. The proximal end 24b includes circumferential protrusions 30, for facilitating attachment to a vacuum hose (a flexible hose or the like connected to a vacuum source, that is not shown in the drawing figures) or other vacuum source (not shown).

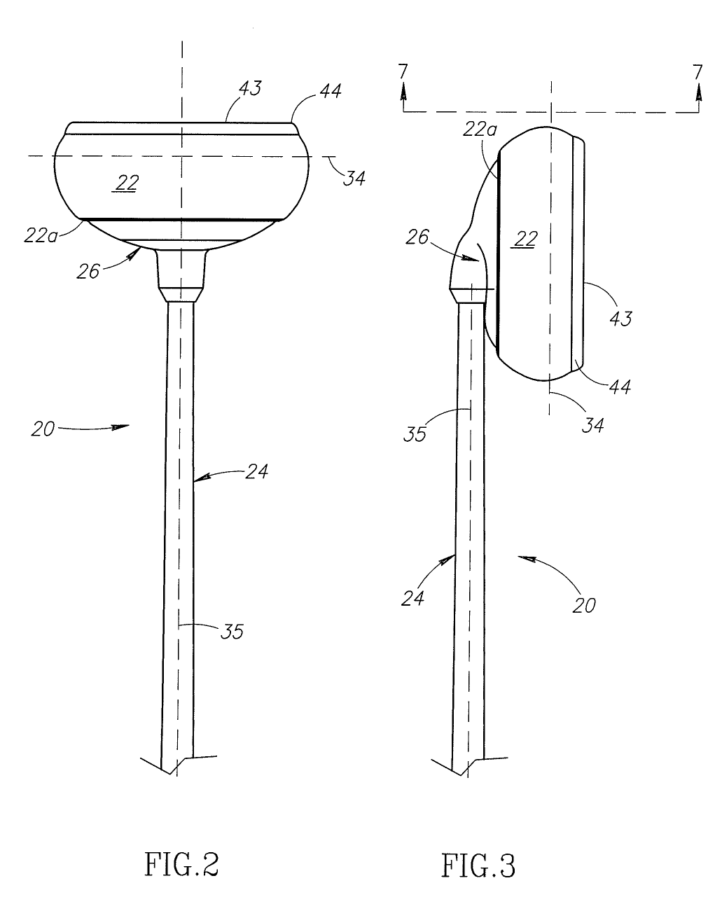

Turning also to FIGS. 2 and 3, there is shown the cup 22 and stem 24, as coupled by the joint 26, such that the cup 22 moves with respect to the stem 24 from a straight position, as shown in FIG. 2, to a bent position, as shown in FIG. 3, and vice versa, as well as all positions there between. In the straight position, the axis 34 of the cup 22 is perpendicular to the axis 35 of the stem 24, while in the bent position, the axis 34 of the cup 22 is parallel to the stem 24. When moving between the straight and bent positions, the joint 26 flexes, allowing for the physician or operator to move the stem 24 to the desired angles (formed by the cup axis 34 and the stem axis 35) between those of the straight and bent positions, to manipulate the apparatus 20.

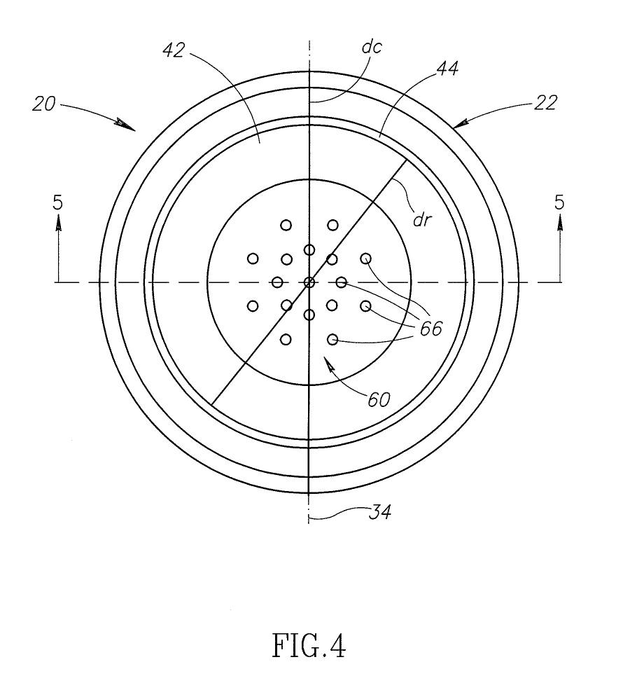

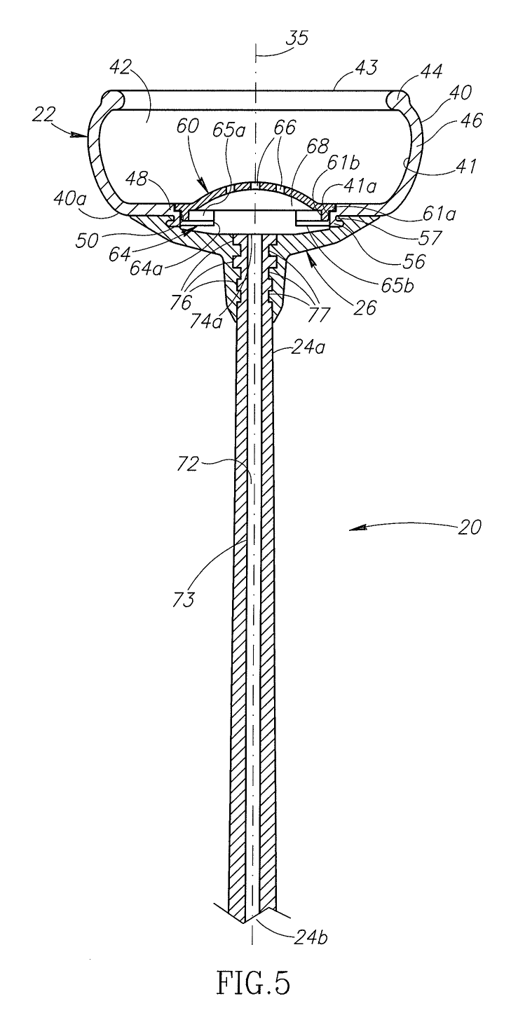

Turning also to FIGS. 4 and 5, the cup 22 is, for example, in a mushroom or bowl-like shape. For example, the mushroom or bowl-like shape is similar to the shape of the cup disclosed in U.S. Pat. No. 5,019,086. While this mushroom shape is preferred, other shapes, such as bell-like, are also suitable, provided that they function suitably for vacuum extraction.

The cup 22 includes an exterior surface 40 and an interior surface 41, the interior surface partially enveloping an interior chamber 42. The cup 22 has a wide or open end 43, formed by a rim 44, that contacts the head of the child. The rim 44, for example, has a rounded contour, and is typically circular or rounded in cross-section. This shape of the rim 44 facilitates gentle gripping of the head of the child, typically around the scalp, so as to maintain a tight seal between the cup 20 and the scalp, suitable to maintain the vacuum in the interior chamber 40, and provide traction between the cup 20 and the scalp, without causing trauma to the scalp. The diameter of the rim 44, expressed as “dr” is less than the diameter of the cup 22, expressed as “dc” along the axis 34 extending through the midpoint of the cup 22.

The cup 22, typically has walls 46 of a thickness sufficient to maintain the integrity of the cup 22 under vacuum pressures as great as 600 mm Hg. The cup 22 may also include protrusions, such as ribs, along its interior surface 41 (within the interior chamber 42), to provide additional structural integrity to the cup 22.

Opposite the wide end 43, proximate the base 22a of the cup 22 is a narrow or base end 48, where the cup 22 couples to the stem 24. The base end 48 includes an opening 50, that has an exterior surface 40a common with the exterior surface 40 of the cup 22, and an interior surface 41a, common with the interior surface 41 of the cup 22. The exterior surface 40a forms a notch 56, typically circumferential around the cup base opening 50, with a circumferential groove 57 therein, to create a secure engagement with the joint 26.

The interior surface 41a is in an L-like shape, extending circumferentially, to receive a filter disc 60, whose peripheral outer edge 61a is correspondingly L-shaped to fit snugly into the opening 50. A cap 64, including a central opening 64a seats snugly on the filter disc 60, as a first portion 65a abuts the peripheral inner edge 61b of the of filter disc 60, and a second portion 65b abuts the inner surface 41a of the opening 50.

The filter disc 60 includes vent openings 66, through which the vacuum is pulled. These vent openings 66 are of a size sufficient to allow for pulling of the vacuum but small enough to prevent large material, such as hair, fetal and maternal tissue, from entering the interior chamber 72 of the stem 24. The vent openings 66, coupled with the central opening 64a of the cap 64, provide for the transfer of the vacuum between the interior chamber 42 of the cup 22 to the interior chamber 72 of the stem 24.

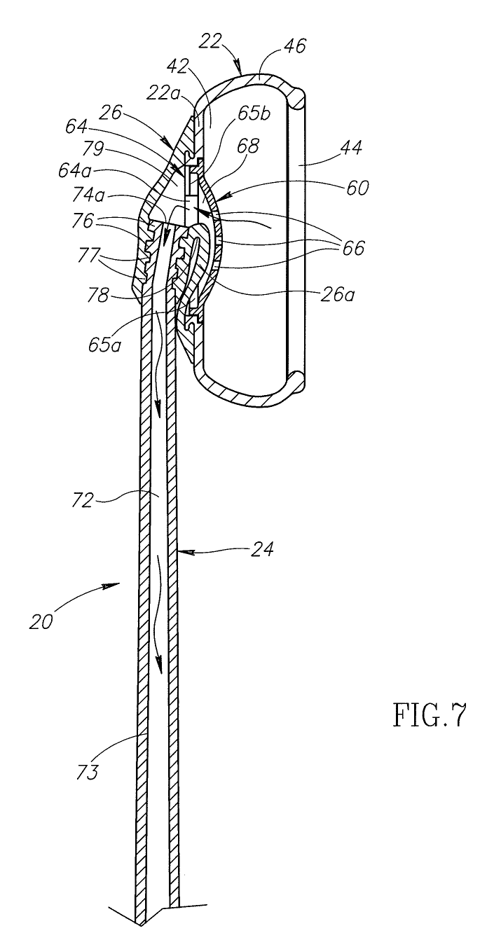

The filter disc 60 is typically rounded, and extends into the interior chamber 42 of the cup 22, with a cavity 68 between the filter disc 60 and the cap 64. This rounded shape of the filter disc 60, coupled with the cavity 68, provides space to accommodate the material of the joint 26 and the cap 64, when the apparatus 20 is in a bent position, and the vacuum is maintained (the vacuum flow indicated by the arrows, that are unlabeled and show direction, hereinafter “directional arrows”), for example, as shown in FIG. 7.

The cup 22 is made of, for example, of a semi-rigid plastic, such as DOW Polyethylene 722, a low density polyethylene (LDPE), from The Dow Chemical Company, Midland, Mich. The cup 22 is typically an integral piece, and is made, for example, by techniques such as injection molding or other conventional plastics manufacturing techniques. The semi-rigid plastic allows the cup 22 to maintain its shape under vacuum.

The filter disc 60 and cap 64 are typically also made, for example, of semi-rigid plastic, such as DOW Polyethylene 722, from The Dow Chemical Company, Midland, Mich., and are typically of the same material as the cup 22. These components typically each integral pieces, and are made, for example, by conventional plastic manufacturing techniques, such as injection molding and the like. The filter disc 60 and cap 64 may be frictionally fit into the cup 22, but are typically secured or to the cup 22 by adhesives, mechanical fasteners, or other bonding techniques such as welding, molding and the like.

The stem 24 has a hollow inner interior chamber 72. The stem 24 typically tapers outward from the distal end 24a to the proximal end 24b, and is, for example, of walls 73 of substantially uniform wall thickness. The wall thickness is such that the structural integrity of the stem 24 can be maintained at the pressures detailed for the cup 22, above. The stem 72 extends from an opening 74a at the distal end 24a to an opening 74b at the proximal end 24b. The opening 74a couples with opening 64a of the cap 64 (and accordingly, the opening 50 of the cup 22 at the base end 48), to serve as a pathway for the vacuum. The coupling is typically coaxial, when the cup 22 is in the straight position, with respect to the stem 24. The opening 74b couples to the vacuum source (not shown). The distal end 24a includes ridges 76, typically circumferential and integral with the stem 24, with grooves 77 between the ridges 76. When coupled with the joint 26, material from the joint 26 enters the grooves 77, to form a strong yet flexible connection between the stem 24 and the joint 26.

The stem 24 is typically made of, for example, of semi-rigid plastic, such as, DOW Polyethylene 722, from The Dow Chemical Company, Midland, Mich., and is typically of the same material as the cup 22. The stem 24 is typically a unitary piece and made, for example, by conventional plastic manufacturing techniques, such as injection molding and the like.

The handle 28 is typically made of a plastic material. It is typically a unitary member and is attached to the stem by conventional techniques such as friction fits, adhesives, welding, and combinations thereof. Alternately, the handle 28 and stem 24, may be made as a single unit, for example, by molding, such as injection molding or the like.

The joint 26 is flexible, and attaches to the cup 22 at the base 22a along the exterior surface 40. The joint 26 is such that the attachment includes material of the joint 26 in the groove 57 of the notch 56 of the exterior surface 40a. The joint 26 attaches to the distal end 24a of the stem 24 and typically extends beyond (proximally) the proximal most ridge 76 and/or groove 77 of the stem 24. Material of the joint 26 is in the grooves 77, for a secure attachment of the stem 24 and the joint 26.

The joint 26, is for example, of a rubber material, such as Kraton® rubber polymer, for example, Kraton® D2109-2. This rubber material is of greater flexibility than the material of the cup 22 and the stem 24. The joint 26 is such that it returns to its original shape without distortion, cracks or creases. Moreover, the joint 26 forms a smooth cover and smooth area between the cup 22 and the stem 24, and does not crease when bent or folded, leaving smooth and non-sharp surfaces to contact maternal tissues.

Turning also to FIG. 7, the joint 26 is of a thickness, such that when the apparatus 20 is in a bent position, for example, about a pinch point 78, a portion 26a of the joint 26 enters into the internal cavity 68 between the filter disc 60 and cap 64, and is accommodated therein, with the vacuum maintained (the vacuum flow indicated by the directional arrows). Additionally, the joint 26 is flexible so as to form an air gap 79 between the cap 64 of the cup 22 and the stem 24, when the stem 24 is the bent, or in an otherwise angled position.

As a result of this material, the joint 26 allows for the smooth and comfortable flexing between the straight and bent positions, and all positions therebetween, as detailed above. This allows for the cup 22 to move about the stem 24, and vice versa, and conform to the axis of the vagina during initiation of the vacuum extraction procedure, and allows the cup 22 and stem 24 to revert to the straight position gradually, as the vaginal walls converge, as the procedure continues. The joint 26 is of a thickness and flexibility, such that when it bends, as shown for example in FIG. 7, the internal chamber 72 of the stem 24, the opening 64a of the cap 64, and the cavity 68, between the filter disc 60 and the cap 64, remain open and form a vacuum flow path (indicated by the directional arrows) with the vent openings 66 of the filter disc 60. This open flow path allows the vacuum to be maintained and pulled from the interior chamber 42 of the cup, by the vacuum source (not shown).

The joint 26 is typically attached to the cup 22 and stem 24 by techniques such as molding. For example, the cup 22 and stem 24 can be separate pieces or a single unitary piece, manufactured previously. The filter disc 60 and cap 64 are placed on the cup 22, as detailed above.

The cup 22 and stem 24 are then placed into an molding system, machine or the like, and the joint 26 is molded over the base 22a of the cup 22 and the distal end 24b of the stem 24. During the molding process, the material of the joint 26 flows into the groove 57 of the notch 56 of the cup 22, and the grooves 77 of the stem 24, creating a strong attachment that will prevent the cup 22 and stem 24 from separating during use. Additional securement of the joint 26 to the cup 22 and stem 24 may be with adhesives, bonding agents, mechanical fasteners and the like.

Attention is again directed to FIGS. 1-7, to illustrate an exemplary operation of the apparatus 20. Initially, the apparatus 20 is inserted into the birth canal, in the bent position of FIG. 7, where the axis 34 of the cup 22 is parallel to the axis 35 of the stem 24. The head of the child is contacted and the requisite vacuum for the desired traction is applied. When the apparatus 20 is in this folded or bent position, the smoothness and lower profile of this apparatus 20 makes positioning of the apparatus 20 easier, because of the lack of sharp corners or creases.

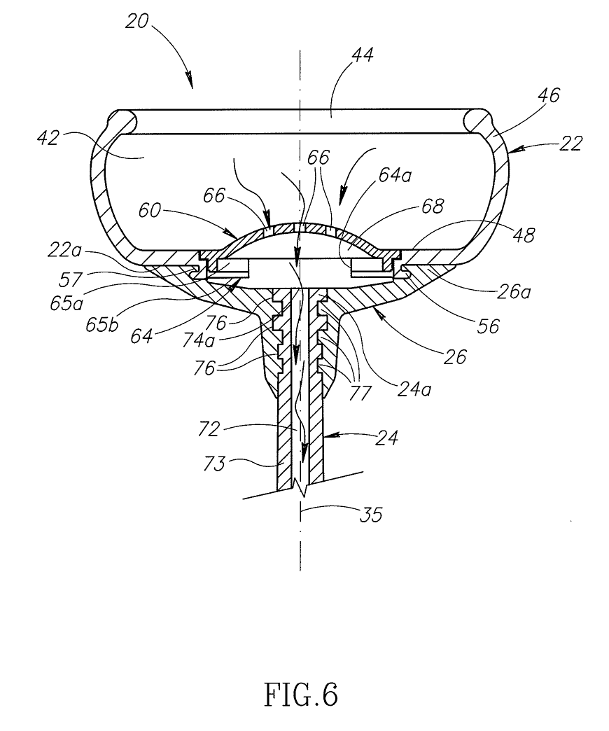

The apparatus 20 may now be manipulated under vacuum (the vacuum indicated by the directional arrows), through various degrees of bending (where the axes 34, 35 are angled at less than a right angle with respect to each other), between the folded or bent position (FIGS. 3 and 7—where the axes of the cup 34 and stem 35 are parallel), and the straight position (FIGS. 2 and 6—where the axes of the cup 34 and stem 35 are perpendicular). The cup 22 can continue to conform to the axis of the vaginal walls, with the vacuum maintained during the various degrees of bending. By allowing this variable bending with the maintenance of the vacuum, the apparatus 20 can correct for asynclitism. Manipulation of the apparatus 20 under vacuum may continue until the child has been sufficiently extracted.

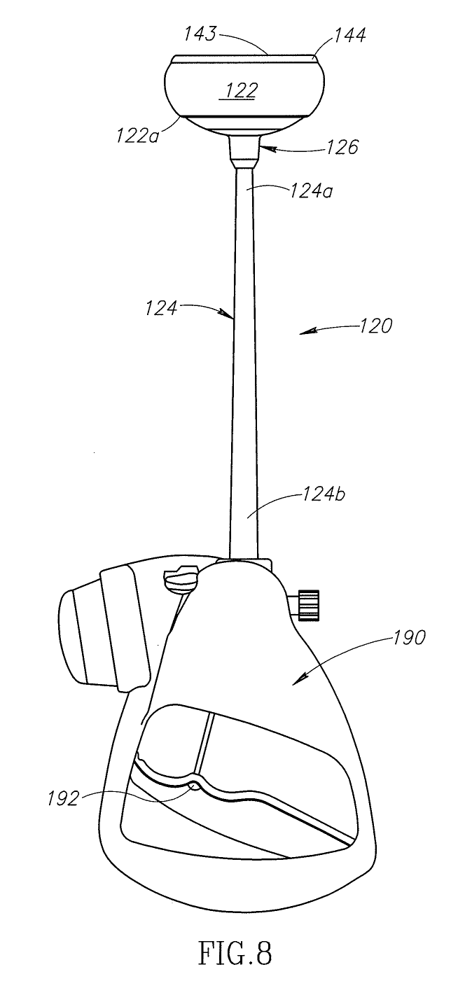

Turning now to FIG. 8, there is shown an alternate apparatus 120 in accordance with the disclosed subject matter. For clarity, the components of the apparatus 120, that have been previously discussed above, with regard to the apparatus 20, and shown in FIGS. 1-7 above, are identified using the same element numbers increased by “100”. The descriptions of these elements are in accordance with the corresponding descriptions above. Different structures as well as differences between the apparatus 20, 120 are described below.

In the apparatus 120, the cup 122 and stem 124 are similar in all aspects of construction and materials to the cup 22 and stem 24 of the apparatus 20. However, the circumferential protrusions 30 are not present at the proximal end 124b of the stem 124, and the handle 28 is not present.

Instead, the proximal end 124b of the stem 124 mounts to a hand pump 190. A handle 192, pumped by manual gripping thereof, generates the requisite vacuum. The hand pump 190 mounts to the stem 124, such that the hand pump 190 is rotatable about the axis 135 of the stem 124, independent of the rotation of the cup 122 and stem 124. This allows the hand pump 190 to be maneuvered about the stem 124, without putting torque on the head of the child being extracted.

While preferred embodiments of the disclosed subject matter have been described, so as to enable one of skill in the art to practice the disclosed subject matter, the preceding description is intended to be exemplary only. It should not be used to limit the scope of the disclosure, which should be determined by reference to the following claims.

1. A vacuum extractor apparatus comprising:
a cup with a first open end and a second open end;
a stem including a first end, for coupling with the second end of the cup, and, a second end for coupling with a vacuum source; and,
a flexible joint for coupling the cup to the stem, the flexible joint configured for bending, to move at least one of the cup or the stem, and configured to accommodate the bending while maintaining a pathway for the vacuum through the apparatus.

2. The vacuum extractor apparatus of claim 1, wherein the cup is mushroom shaped.

3. The vacuum extractor apparatus of claim 1, wherein the cup and the stem each include an axis extending therethrough, and the joint accommodates the cup and the stem at angular orientations of the axes between positions where the axis of the cup is at least substantially perpendicular to the axis of the stem and where the axis of the cup is at least substantially parallel to the axis of the stem, and, maintains a pathway for a vacuum through the apparatus.

4. The vacuum extractor apparatus of claim 1, wherein the cup is of a first material and the flexible joint is of a second material of greater flexibility than the first material.

5. The vacuum extractor apparatus of claim 1, additionally comprising: a handle positioned along the second end of the stem.

6. The vacuum extractor of claim 1, wherein the flexible joint includes an outer surface that forms a smooth transition with the outer surfaces of cup.

7. A vacuum extractor apparatus comprising:
a cup with a first open end and a second open end;
a stem including a first end in communication with the second end of the cup, and, a second end for communication with a vacuum source; and,
a joint for coupling the cup to the stem, the joint configured for accommodating bending of the apparatus at the joint at least at a pinch point, and configured to accommodate the bending while maintaining a vacuum through the apparatus.

8. The vacuum extractor apparatus of claim 7, wherein the cup is mushroom shaped.

9. The vacuum extractor apparatus of claim 7, wherein the cup and the stem each include an axis extending therethrough, and the joint accommodates the cup and the stem at angular orientations of the axes, and, maintains a vacuum through the apparatus.

10. The vacuum extractor apparatus of claim 7, wherein the cup is of a first material and the flexible joint is of a second material of greater flexibility than the first material.

11. The vacuum extractor apparatus of claim 7, additionally comprising: a handle positioned along the second end of the stem.

12. The vacuum extractor apparatus of claim 7, additionally comprising: a vacuum source including a hand pump for producing a vacuum.

13. The vacuum extractor of claim 12, wherein the hand pump is in communication with the stem so as to rotatable independent of the stem.

14. A vacuum extractor apparatus comprising:
a cup with a first open end and a second open end;
a stem including a first end in communication with the second end of the cup, and, a second end for communication with a vacuum source; and,
a joint for coupling the cup to the stem, the joint configured for accommodating bending of the apparatus at the joint at least at a pinch point, and configured to bend such that its outer surface remains smooth.

15. The vacuum extractor apparatus of claim 14, wherein the joint is additionally configured for bending while maintaining a vacuum through the apparatus.

16. The vacuum extractor apparatus of claim 14, wherein the cup is mushroom shaped.

17. The vacuum extractor apparatus of claim 14, wherein the cup and the stem each include an axis extending therethrough, and the joint accommodates the cup and the stem at angular orientations of the axes, and, maintains a vacuum through the apparatus.

18. The vacuum extractor apparatus of claim 14, wherein the cup is of a first material and the flexible joint is of a second material of greater flexibility than the first material.

19. A method for making a vacuum extractor apparatus comprising:
providing a cup and a stem in communication with the cup at least substantially coaxial; and,
molding a joint of a flexible material onto the cup and the stem.

20. The method of claim 19, wherein the cup and the stem are a unitary member.

21. The method of claim 19, wherein the cup and the stem are separate pieces.

22. The method of claim 19, wherein the cup is of a semi rigid material of less flexibility than the flexible material of the joint.

23. A method for assisting in the extraction of a child during childbirth, comprising:
providing an extraction apparatus comprising:
a cup with a first open end and a second open end;
a stem including a first end in communication with the second end of the cup, and, a second end for communication with a vacuum source; and,
a joint for coupling the cup to the stem, the joint configured for accommodating bending of the apparatus at the joint at least at a pinch point, and configured to accommodate the bending while maintaining a vacuum through the apparatus;
connecting the stem to a vacuum source;
moving the stem to cause the joint to bend;
inserting the cup into the birth canal into contact with the head of a child;
activating the vacuum source; and,
moving the stem to maneuver the child through the birth canal.

24. The method of claim 23, wherein the vacuum source includes a hand pump in communication with the stem so as to be rotatable independent of the stem.

25. The method of claim 24, additionally comprising: rotating the hand pump while moving the stem to maneuver the child through the birth canal.
